SEQUENCE LISTING (10 sequences)

SEQ ID NO: 1 - DNA, 45 bp, Artificial Sequence
  synthetic
caagttttcaccgcggtttttctagttaacatgatatcgattccc

SEQ ID NO: 2 - DNA, 29 bp, Artificial Sequence
  synthetic
gagtccgggatttctagatcccggtcgac

SEQ ID NO: 3 - DNA, 40 bp, Artificial Sequence
  synthetic
ccattatttttctagacacccatatggtttttttacctcc

SEQ ID NO: 4 - DNA, 29 bp, Artificial Sequence
  synthetic
caaaagatggatccgctcgtcatgaagag

SEQ ID NO: 5 - DNA, 29 bp, Artificial Sequence
  synthetic
caaaagatggatccgctcgtcatgaagag

SEQ ID NO: 6 - DNA, 33 bp, Artificial Sequence
  synthetic
ccattatttttctagaccatatgcttcacatcc

SEQ ID NO: 7 - DNA, 56 bp, Artificial Sequence
  synthetic
gggggcggccgcacaactttgtatagaaaagttgagaaacgtaaaatgatataaat

SEQ ID NO: 8 - DNA, 56 bp, Artificial Sequence
  synthetic
ggggcgccggcgacaacttttttgtacaaagttgagaaacgtaaaatgatataaat

SEQ ID NO: 9 - DNA, 23 bp, Artificial Sequence
  synthetic
caccatgactcaactgaagccac

SEQ ID NO: 10 - DNA, 18 bp, Artificial Sequence
  synthetic
atagcgagcgaggtgttc

(19) United States
(12) Patent Application Publication
(10) Pub. No.: US 2008/0131925 A1
(43) Pub. Date: Jun. 5, 2008

(54) Production of Beta-Lactams in Single Cells

(76) Inventors: Marco Alexander Van Den Berg, Poeldijk (NL); Roelof Ary Lans Bovenberg, Rotterdam (NL); Hendrik Jan Noorman, Delft (NL); Bastiaan Romein, Delft (NL)

Correspondence Address: NIXON & VANDERHYE, PC, 901 NORTH GLEBE ROAD, 11TH FLOOR, ARLINGTON, VA, 22203, US

(21) Appl. No.: 11/792,725

(22) PCT Filed: Dec. 8, 2005
(86) PCT No.: PCT/EP05/56619

§ 371 Date: Jun. 11, 2007

(30) Foreign Application Priority Data
Dec. 10, 2004 (EP) 04106471.8

(51) Int. Cl. C12P 37/00; C12N 1/00

(52) U.S. Cl. 435/43; 435/243; 435/254.2; 435/254.21; 435/254.22; 435/254.23

(57) ABSTRACT

The present invention describes the transformation of a microorganism that does not naturally produce a β-lactam compound with polynucleotides involved in the biosynthesis of β-lactam compounds and the use of such transformed microorganisms in the production of β-lactam compounds or in the identification of genes or factors involved in the synthesis of a β-lactam compound.

The present invention relates to a process for the production of a β-lactam compound and to cells that can be used in such production.

β-Lactam compounds currently are produced on a commercial scale by filamentous microorganisms, such as Penicillium chrysogenum, Streptomyces clavuligerus, Nocardia lactamdurans and Acremonium chrysogenum, as endogenous secondary metabolites.

Examples of microbial produced β-lactam compounds are penam compounds, such as penicillin V, (iso) penicillin N and penicillin G, cephem compounds such as desacetoxycephalosporin and other acyl-7-aminodesacetoxycephalosporanic acids, desacetylcephalosporanic acid and other acyl-7-aminodesacetylcephalosporanic acids, cephalosporin C and other acyl-7amino-cephalosporanic acids, clavam compounds such as clavulanic acid, carbapenem compounds, such as imipenem and thienamycin and monobactam compounds such as aztreonam.

Examples of natural β-lactam-producing organisms are Aspergillus (A. nidulans), Acremonium (A. chrysogenum), Erwinia (E. carotovora), Flavobacterium, Kallichroma (K. tethys), Nocardia (N. lactamdurans, N. uniformis), Penicillium (P. chrysogenum, P. nalgiovense, P. griseofulvum) and Streptomyces (S. antibioticus, S. cattleya, S. clavuligerus, S. griseus, S. hygroscopicus, S. lipmanii).

The production level of the β-lactam compounds in the commercially applied organisms has been increased considerably over the years. For instance, a modern Penicillium chrysogenum production strain is reported to produce about 40-50 g/l, whereas the original strains produced about 1 mg/l (Elander, R. P. (2002) University of Wisconsin contributions to the early development of penicillin and cephalosporin antibiotics, SIM News 52, 270-278; Elander, R. P. (2003) Industrial production of β-lactam antibiotics, Appl Microbiol Biotechnol 61, 385-392).

This enhanced production level was realized by classical mutagenesis techniques (Elander, R. (1983) Strain improvement and preservation of β-lactam producing microorganisms. In A. L. Demain and N. Solomon (eds.) Antibiotics containing the β-lactam structure I, Springer-Verlag, New York, N.Y., 97-146).

Furthermore, application of recombinant DNA techniques resulted in production of cephem β-lactams in Penicillium strains that naturally have the ability to produce penam β-lactams only. On the other hand, Acremonium strains, which normally produce cephem compounds, have been genetically modified to produce penam compounds.

However, a serious drawback of the use of microorganisms naturally capable of producing β-lactams is that these organisms generally are filamentous organisms during their production phase. This filamentous nature poses serious difficulties during the fermentation. The actual rheology is highly dependent on culture conditions and can change from pellet growth to viscous growth. The former causing nutrient transport problems, the latter causing limitations in oxygen transfer. Furthermore, the individual compartments of the hyphae are differentiating during the fermentation: from the growing apical or tip cell (with the so-called ‘spitzenköorper’ as point of new growth), the young and healthy-looking subapical cells, and the old and highly vacuolized cells. These different cells may differ in production levels and respond differently to culture conditions, hampering process control.

In addition, investigation directed to factors that are involved in and/or influence β-lactam production is not feasible using these filamentous, naturally β-lactam producing organisms. First, compared to organisms like Escherichia coli they are poorly accessible to standard molecular biology techniques. Transformation is via fragile protoplasts in polyethylene glycol, complete episomally maintained plasmids are not available, and integration is mostly random and multicopy. On top of that the hyphal compartments are multi-nucleate and the most important species, P. chrysogenum, has no sexual cycle and also the alternative, the parasexual cycle, is very inefficient. All these factors severely hamper the identification of essential genes.

For this reason there is a desire to have β-lactam-producing organisms that are better suited for large-scale production and that do not have the disadvantageous properties typical for filamentous microorganisms. In addition, there is a desire to avail of microorganisms wherein factors involved in β-lactam production can be more easily investigated.

Up to now, the establishment of β-lactam production in non-filamentous, microbial single cells was not feasible due to several factors.

The first committed step in β-lactam synthesis is catalyzed by the so-called Non Ribosomal Peptide Synthetase class of enzymes, in this case δ-(L-α-aminoadipyl)-L-cysteinyl-D-valine synthetase (ACVS). These modular enzymes catalyze a complex series of amino acid activation and subsequent peptide bond formation. Species like bakers' yeast (S. cerevisiae) do not have such enzymes, therefore might not be equipped to perform such a reaction. In addition, the gene encoding ACVS, pcbAB, is approximately 12 kb long. With promoter and terminator, an expression cassette of 14 kb needs to be stably integrated in the yeast genome.

The second step in β-lactam synthesis is one of unprecedented, complex biochemistry, a non-heme oxidation by isopenicillin N synthase (IPNS). This enzyme is severely inhibited by many standard compounds in the cell (glutathion, Mn, Zn, pH changes, etc). Yeast has quite high glutathion levels under certain growth conditions, possibly causing severe inhibition of the IPNS enzyme.

The third step in β-lactam synthesis, catalyzed by 6-APA:AcylCoA acyl transferase (AT), is encoded by a gene interrupted by three fungal introns. As yeast has only few genes with introns, which are also different from fungal introns, these need to be removed to obtain proper expression in yeast. In addition, AT is only functional as a heterologous dimer. The two components are both derived by auto-processing from the initial polypeptide encoded by the gene, generating 10 and 29 kDa peptides. It is not known if this auto-processing will work in yeast cells.

β-Lactam enzymes are notorious for their instability and the environment in filamentous fungi is equipped to regenerate continuously with new enzymes to support the continuous production of β-lactams. It is not known beforehand if yeasts also have this regenerating ability.

Natural β-lactam producers have evolved secretion systems specifically adapted for efficient β-lactam export to sustain a high production level.

Last but not least, β-lactams are toxic products. Non-natural producers may not be equipped to survive these compounds.

According to the present invention it has surprisingly been found that β-lactam production can be established in a microorganism that does not naturally produce a β-lactam compound, i.e. a microorganism that does not possess the biosynthetic pathway leading to the formation of a β-lactam compound. Thus, in a first aspect the present invention provides a microorganism that does not naturally produce a β-lactam compound and that is transformed with a polynucleotide involved in the production of a β-lactam compound.

Preferably, this microorganism that does not naturally produce a β-lactam compound grows in the form of single cells, more preferably is a eukaryotic microorganism that grows in the form of single cells. Most preferably, the microorganism that does not naturally produce a β-lactam compound is a yeast.

Suitable yeasts to be used in the present invention are Saccharomyces (S. cerevisiae, S. bayanus, S. exiguus), Candida (C. glabrata, C. utilis, C. maltosa, C. albicans, C. boidinli, C. tropicalis), Kluyveromyces (K. lactis, K. marxianus, K. thermotolerans), Yabadazyma ohmeri, Pichia (P. angusta (=Hansenula polymorpha), P. sorbitophila), Yarrowia lipolitica, Zygosaccharomyces rouxii.

The term “single cells” as used in the context of the present invention refers to microorganisms that grow predominantly or solely in the form of single cells, i.e. microorganisms that do not predominantly or solely grow in the form of hyphae, pellets and/or as filamentous organisms. This advantageously allows cultivation of the microorganism to a much higher cell density than would be possible with filamentous organisms.

Sometimes, the growth behavior of a microorganism that naturally grows as a single cell may be changed due to e.g. genetic modification. For instance, modifications are known that cause budding problems in yeast. The skilled person will understand that such a modified organism that may not necessarily grow solely in the form of single cells still is within the scope of the present invention.

The present invention has several advantages: a reduced viscosity of the fermentation broth, e.g. a lower stirrer speed suffices to ensure appropriate mixing of the fermentation broth, the possibility of obtaining a higher oxygen transfer rate in the fermentor, thereby allowing an increased feed rate of the carbon source to the fermentation, the possibility to obtain a higher carbon flux through the β-lactam pathway, no differentiation between cells causing all cells to be producing cells.

A polynucleotide involved in the production of a β-lactam compound comprises a polynucleotide sequence encoding an enzyme of the β-lactam biosynthetic pathway. Examples of enzymes that are part of the β-lactam biosynthetic pathway are:
(Tri) peptide synthetases such as δ-(L-α-aminoadipyl)-L-cysteinyl-D-valine synthetase (ACVS), e.g. encoded by the pcbAB gene of Penicillium chrysogenum,
Non-heme iron-containing dioxygenases such as isopenicillin N synthase (IPNS), e.g. encoded by the pcbC gene of Penicillium chrysogenum,
Epimerases such as IPN epimerase, e.g. encoded by the cefD gene of Streptomyces clavuligerus,
Acyl transferases such as 6-APA:AcylCoA acyl transferase (AT), e.g. encoded by the penDE gene of Penicillium chrysogenum,
Expandases such as desacetoxycephalosporin C synthase (DAOCS) comprising the enzyme encoded by the cefEF gene of Acremonium chrysogenum or the cefE gene of Streptomyces clavuligerus,
Hydroxylases such as desacetylcephalosporin C synthase comprising enzyme encoded by the cefEF gene of Acremonium chrysogenum or the cefF gene of Streptomyces clavuligerus,
Transferases such as O-carbamoyl transferase (CAT), e.g. encoded by the cmcH gene of Streptomyces clavuligerus.

Preferably, the polynucleotide involved in the production of a β-lactam compound further comprises a polynucleotide sequence encoding a protein that has a supportive function in the production of a β-lactam compound by a microorganism that does not naturally produce a β-lactam compound. With a supportive function is meant that the protein is not an enzyme that is part of the biosynthetic pathway of a β-lactam compound, but that the protein is necessary for efficient β-lactam production. Necessary for efficient β-lactam production means that the protein may be essential for β-lactam production, i.e. when absent no β-lactam production is measurable in the microorganism, even when all relevant biosynthetic enzymes are present, and/or may be necessary to obtain a suitable β-lactam production level, i.e. when absent a too low β-lactam production is measurable in the microorganism. Examples of proteins that have a supportive function are:
Regulatory proteins of the β-lactam biosynthetic pathway, such as cpcRI (Schmitt, EK. and Kuck, U (2000) The fungal CPCR1 protein, which binds specifically to beta-lactam biosynthesis genes, is related to human regulatory factor X transcription factors, J Biol Chem. 275:9348-9357); claR (Perez-Redondo R, Rodriguez-Garcia A, Martin J F, Liras P. (1998) The claR gene of Streptomyces clavuligerus, encoding a LysR-type regulatory protein controlling clavulanic acid biosynthesis, is linked to the clavulanate-9-aldehyde reductase (car) gene, Mol. Microbiol. 27:831-843); ccaR (Perez-Llarena F J, Liras P, Rodriguez-Garcia A, Martin J F. (1997) A regulatory gene (ccaR) required for cephamycin and clavulanic acid production in Streptomyces clavuligerus: amplification results in overproduction of both β-lactam compounds, J. Bacteriol. 179: 2053-2059),
Transporter proteins that deliver precursors of the β-lactam biosynthetic pathway to the appropriate site in the cell, such as ATP-type Binding Cassette (ABC) proteins like aa1, aa5, aa7, aa10, dd2 (WO 01/32904) and such as Multi-Facilitator Superfamily (MFS) proteins like cefT (Ullán, R. V., Liu, G., Casqueiro, J., Gutierrez, S., Banuelos, 0. & Martin, J. F. (2002) The cefT gene of Acremonium chrysogenum encodes a putative multidrug efflux pump protein that significantly increases cephalosporin C production. Mol Genet Genomics 267, 673-683),
Enzymes involved in primary metabolism, especially involved in the formation of primary metabolites that are precursors for cysteine, such as oasS, encoding O-acetyl-L-serine sulfhydrylase (WO 99/01561); and for aminoadipate, such as for lysine (Casqueiro J, Gutierrez S, Banuelos 0, Hijarrubia M J, Martin J F. (1999) Gene targeting in Penicillium chrysogenum: disruption of the lys2 gene leads to penicillin overproduction, J Bacteriol. 181:1181-1188),
Enzymes necessary for activation of the side chain, such as phenyl acetyl coenzyme A ligase, encoded by pcl (WO 97/02349),
Enzymes involved in the activation of amino acids by ACVS, such as the Aspergillus nidulans phosphopantenoyl transferase encoded by npgA (Keszenman-Pereyra D, Lawrence S, Twfieg M E, Price J, Turner G. (2003) The npgA/cfwA gene encodes a putative 4′-phosphopantetheinyl transferase which is essential for penicillin biosynthesis in Aspergillus nidulans, Curr Genet. 43:186-190),
Enzymes involved in peroxisome proliferation like Pex11P, encoded by pex11 (WO 00/71579).

Apart from the naturally occurring genes encoding β-lactam biosynthetic enzymes or naturally occurring polynucleotides encoding proteins with a supportive function use can be made also of polynucleotide sequences encoding artificial mutants of these enzymes or supportive proteins. Such mutants may show greater stability, enhanced performance (such as an enhanced activity or a different localization) or different specificity as compared to the native enzymes or proteins.

The microorganism that does not naturally produce a β-lactam compound but wherein β-lactam is to be established according to the invention may conveniently be prepared according to methods commonly known in the art.

Briefly, a polynucleotide involved in β-lactam production can be incorporated into a suitable vector. Such a vector can be a circular or linear vector. In addition, the vector may provide episomal replication, i.e. replication of the vector outside the genomic DNA of the cell, or may necessitate integration of the polynucleotide into the genome. Preferably, the vector is an expression vector, providing for expression of the polynucleotide involved in β-lactam production in the microorganism wherein β-lactam is to be established. The coding sequence of the polynucleotide involved in β-lactam production is provided with regulatory sequences ensuring expression of the encoded polypeptide. The regulatory sequences may be the ones naturally associated with the coding sequence in question or may be sequences selected for their capability to ensure suitable expression in the microorganism of choice. Polynucleotides involved in β-lactam production may be incorporated into one vector or into separate vectors for each different polynucleotide. If two or more polynucleotides involved in β-lactam production are combined, it is possible to provide each polynucleotide with an individual regulatory region (polycistronic organization) or to use one regulatory region for the two or more polynucleotides (according to the so-called monocistronic or operon structure). It is also possible to combine certain polynucleotides in one vector and use separate vectors for other polynucleotides.

Transformation methods for introduction of a polynucleotide into a microorganism of choice are commonly available for various types of microorganisms.

In particular, yeast cells can be transformed by first providing for different yeast cell populations each transformed with one of the desired polynucleotides, and subsequent crossing over of the respective transformed yeast cell populations thus obtaining a population of yeast cells containing all of the desired polynucleotides. Alternatively, once transformed, yeast cells can be retransformed using either a different selection marker or the same, upon removal of the marker by the available systems (e.g. cre-lox, FLP-FRT, see for reviews Gilbertson L. (2003) Cre-lox recombination: Creative tools for plant biotechnology, Trends Biotechnol. 21:550-555; Luo H, Kausch A P (2002) Application of FLP/FRT site-specific DNA recombination system in plants, Genet Eng (NY). 24:1-16).

In one embodiment one should place the P. chrysogenum pcbAB, pcbC, penDE and pc genes (encoding ACVS, IPNS, AT and PCL, respectively) under control of a S. cerevisiae specific promoter like the MET25-promoter and a S. cerevisiae specific terminator like the MET25-terminator; and integrate the separate expression cassettes into the yeast genome. The determination of enzyme activities of these four different enzymes is done according to methods known in the art: antibodies may be used to detect the presence of the protein and specific assays are used for determining the specific activity of the enzyme. Examples of such assays can be found in Thellgaard H, van Den Berg M, Mulder C, Bovenberg R, Nielsen J. (2001) Quantitative analysis of Penicillium chrysogenum Wis54-1255 transformants over expressing the penicillin biosynthetic genes, Biotechnol Bioeng 72:379-388. (For ACVS); Thellgaard et al (2001) (for IPNS); Tobin M B, Fleming M D, Skatrud P L, Miller J R. (1990) Molecular characterization of the acyl-coenzyme A:isopenicillin N acyltransferase gene (penDE) from Penicillium chrysogenum and Aspergillus nidulans and activity of recombinant enzyme in Escherichia coli. J Bacteriol. 172:5908-5914 (for AT) and Minambres B, Martinez-Blanco H, Olivera E R, Garcia B, Diez B, Barredo J L, Moreno M A, Schleissner C, Salto F, Luengo J M. (1998) Molecular cloning and expression in different microbes of the DNA encoding Pseudomonas putida U phenyl acetyl-CoA ligase. Use of this gene to improve the rate of benzyl penicillin biosynthesis in Penicillium chrysogenum. J Biol Chem. 271:33531-33538 (for PCL).

The production of a β-lactam compound according to the invention or of a β-lactam intermediate like ACV or IPN may conveniently be determined by for instance LC-MS based assays.

A second aspect of the invention concerns a process for the production of a β-lactam compound using the microorganism of the first aspect. The process comprises cultivating the microorganism of the first aspect under conditions conducive to the production of said β-lactam compound. The cultivation conditions are not critical to the invention, provided that a β-lactam compound is produced. Commonly known culture media and conditions can be used. The skilled person will easily understand that the type of β-lactam compound that is produced will depend on the biosynthetic genes that are expressed in the microorganism of the first aspect.

The β-lactam compound preferably is penicillin G, penicillin V, adipoyl-7-aminodesacetoxy cephalosporanic acid (adipoyl-7-ADCA) or adipoyl-7-amino-3-carbamoyloxymethyl-3-cephem-4-carboxylic acid (adipoyl-7-ACCA). Optionally, the process further comprises a deacylation at position 6 if the β-lactam compound is a penam or at position 7 if the β-lactam compound is a cephem, to enable production of e.g. 6-amino-penicillanic acid (6-APA), 7-ADCA or 7-ACCA, respectively.

A third aspect of the invention concerns the use of the microorganism of the first aspect to identify genes and/or factors that influence β-lactam production. This can be done by a range of experiments, for instance:
Growing wild-type S. cerevisiae and β-lactam producing S. cerevisiae under producing and non-producing conditions, comparing the individual responses with available ‘omics’ techniques (e.g. transcriptomics, proteomics, metabolomics, etc),
Challenge wild-type S. cerevisiae and β-lactam producing S. cerevisiae with a range of different β-lactams while growing under relevant conditions and comparing the individual responses with available ‘omics’ techniques (e.g. transcriptomics, proteomics, metabolomics, etc),
Modify the expression of each gene in β-lactam producing S. cerevisiae (knock-outs, under expression and over expression) and analyze the individual responses with available ‘omics’ techniques (e.g. transcriptomics, proteomics, metabolomics, etc).
Express any available gene and/or regulating factor (like non-coding RNA, tRNAs, upstream-regulating RNAs, etc) in β-lactam producing S. cerevisiae and analyze the individual responses with available ‘omics’ techniques (e.g. transcriptomics, proteomics, metabolomics, etc),
Modify the culture conditions (e.g. media compositions, shape of culture vessels and/or feeding-schemes) for wild-type S. cerevisiae and β-lactam producing S. cerevisiae and compare the individual responses with available ‘omics’ techniques (e.g. transcriptomics, proteomics, metabolomics, etc).

The results of these analyses may be advantageously integrated and used to generate leads for further improvement of β-lactam production in β-lactam producing species (including the natural producers like P. chrysogenum, A. chrysogenum and S. clavuligerus).

### EXAMPLES

### Example 1

### Construction of Expression Vectors

The expression vectors were created based on yeast plasmid pRS406 (Sikorski, R. S. and Hieter, P (1989) A system of shuttle vectors and yeast host strains designed for efficient manipulation of DNA in Saccharomyces cerevisiae, Genetics 122:19-27). The Met25 promoter and terminator region (Johnston, M. et al (1997) The nucleotide sequence of Saccharomyces cerevisiae chromosome XII, Nature 387 (6632 Suppl.), 87-90) was isolated from pRS416Met25 (Mumberg D, Muller R, Funk M. (1995) Yeast vectors for the controlled expression of heterologous proteins in different genetic backgrounds. Gene 156:119-122).

Construction of pRS403Met25 and pRS405Met25: pRS416Met25 was restricted with Sstl and Nael resulting in a Met25 promoter and terminator fragment. Subsequent ligation of this fragment into Sstl/Nael digested pRS403 and pRS405 (Sikorski, R. S. and Hieter, P, 1989) results in the desired plasmids.

Construction of pRS404Met25 and pRS406Met25: pRS416Met25 was restricted with Sstl and Kpnl resulting in a Met25 promoter and terminator fragment. Subsequent ligation of this fragment into Sstl/Kpnl digested pRS404 and pRS406 (Sikorski, R. S. and Hieter, P, 1989) results in the desired plasmids.

Plasmid pRS406Met25pcbC (IPNS gene), which was used for integration of the IPNS gene into the yeast chromosome, was prepared by PCR amplification of the P. chrysogenum pcbC gene (Carr, L. G., Skatrud, P. L., Scheetz, M. E. II, Queener, S. W. and Ingolia, T. D. (1986) Cloning and expression of the isopenicillin N synthetase gene from Penicillium chrysogenum, Gene 48:257-266) with the primers

(SEQ ID NO: 1)

5′ CAAGTTTTCACCGCGGTTTTTCTAGTTAACATGATATCGATTCCC-
3′
and

(SEQ ID NO: 2)

5′- GAGTCCGGGATTTCTAGATCCCGGTCGAC-3′,

subsequent cloning in the pCR TOPO2.1 vector (Invitrogen, according to the protocols supplied by the manufacturer), followed by restriction enzyme digestion with Xhol and Spel, and subsequent ligation of the pcbC fragment in a Xhol/Spel restricted pRS406Met25 vector. Construction of the PCL integration plasmid pRS403Met25pcl: pRS403Met25 was restricted with Xbal/BamH1 and ligated with Xbal/BamH1 digested PCR-amplified PCL gene (WO 97/02349), using the primers

(SEQ ID NO: 3)

5′-CCATTATTTTTCTAGACACCCATATGGTTTTTTTACCTCC-3′
and

(SEQ ID NO: 4)

5′-CAAAAGATGGATCCGCTCGTCATGAAGAG-3′.

Construction of the AT (penDE) integration plasmid pRS405Met25penDE: the penDE gene (Tobin et al, 1990) was PCR amplified with the primers

(SEQ ID NO: 5)

5′- CAAAAGATGGATCCGCTCGTCATGAAGAG-3′
and

(SEQ ID NO: 6)

5′- CCATTATTTTTCTAGACCATATGCTTCACATCC-3′

and subsequently restricted with Xbal, BamH1, the resulting penDE fragment was ligated into pRS405Met25 restricted with Xbal, BamH1.

Construction of the ACVS (pcbAB) integration plasmid pRS404DestACVS was as follows. Plasmid pRS404Dest was constructed by first amplifying the CapccdB selection cassette of pDEST15 (Invitrogen) using the primers

| | |
|---|---|
| 5′-GGGGGCGGCCGCACAACTTTGTATAGAAAAGTTGAGAAACGTAAAATGATATAAAT-3′ | (SEQ ID NO: 7) |
| and | |
| 5′-GGGGCGCCGGCGACAACTTTTTTGTACAAAGTTGAGAAACGTAAAATGATATAAAT-3′, | (SEQ ID NO: 8) |

followed by ligation into pCR2.1 TOPO yielding the plasmid pCR2.1/catccdB. The plasmid pCR2.1/catccdB was then digested with Munl and the catccdB fragment was ligated into EcoRI-digested pRS404Met25 to yield pRS404Dest. The pcbAB gene (Diez, B., Gutierrez, S., Barredo, J. L., van Solingen, P., van der Voort, L. H. and Martin, J. F. (1990) The cluster of penicillin biosynthetic genes. Identification and characterization of the pcbAB gene encoding the alpha-aminoadipy-cysteinyl-valine synthetase and linkage to the pcbC and penDE genes, J. Biol. Chem. 265: 16358-16365) was obtained by amplification using the primers

| | |
|---|---|
| 5′-CACCATGACTCAACTGAAGCCAC-3′ | (SEQ ID NO: 9) |
| and | |
| 5′-ATAGCGAGCGAGGTGTTC-3′. | (SEQ ID NO: 10 |

The blunt-ended PCR fragment was cloned into pENTR/SD/D-Topo Vector (Invitrogen), according to the supplier's manual, to yield pENTR/SD/ACVS.

The final integration plasmid pRS404DestACVS was obtained by Gateway LR-Reaction of pENTR-SD-ACVS plasmid with pRS404Dest according to Invitrogen's Gateway manual.

### Example 2

### Transformation of S. cerevisiae

The resulting plasmids carrying the genes that encode for ACVS, IPNS, PCL and AT were transformed into the yeast Saccharomyces cerevisiae CEN-Pk2-1c (Wieczorke R, Krampe S, Weierstall T, Freidel K, Hollenberg C P, Boles E, (1999) Concurrent knock-out of at least 20 transporter genes is required to block uptake of hexoses in Saccharomyces cerevisiae, FEBS Lett. 464:123-128). As a protocol, the high-efficiency yeast transformation procedure based on Lithium-Ion and Herring sperm carrier DNA treatment as described by Gietz R D, Woods R A (2002, Transformation of yeast by lithium acetate/single-stranded carrier DNA/polyethylene glycol method, Methods Enzymol. 350:87-96) was used. Selection of integrants using the auxotrophic markers HIS, LEU, TRP and URA was employed as known in the art. The resulting strain harbored the Penicillin biosynthetic genes encoding ACVS, IPNS, AT and PCL, as judged by PCR and Southern blot analyses.

### Example 3

### Detection of Biosynthetic Enzymes and Enzyme Activity

The putative penicillin production yeast strains were screened for enzyme activities by growth on yeast minimal medium (1×YNB, 20 mM Phosphate pH 6.8, 2% glucose). Under these conditions, the Met25 promoter is fully derepressed due to the absence of methionine. Yeast was grown overnight until a final OD600 of 4-5 was reached. Subsequently, the cells were pelleted and Cell-Free Extract was obtained using sonication or glass beads. The lysate fractions and the soluble supernatant were screened for penicillin biosynthetic enzyme production. Analyses were carried out on Coomassie-stained SDS-PAGE gels and by Western blotting, showing the production of the biosynthetic enzymes. LC-MS was used to demonstrate the formation of the penicillin biosynthesis intermediates ACV, IPN and Pen G.

### Example 4

### Detection of Antibiotic Activity

Colony purified yeast strains were transferred to agar plates that stimulate the production of β-lactam and incubated for 24-168 hours at 25° C. The β-lactam sensitive E. coli ESS strain (Hsu J S, Yang Y B, Deng C H, Wei C L, Liaw S H, Tsai Y C. (2004) Family shuffling of expandase genes to enhance substrate specificity for penicillin G. Appl Environ Microbiol. 70:6257-6263) was cultivated in 2×TY to mid-log phase and diluted in pre-warmed 0.8% 2×TY agar and carefully distributed over the yeast colonies. After incubation at 37 C overnight β-lactam producing yeasts are visible by a cleared zone around the colonies, a so-called halo.

1. A microorganism that does not naturally produce a β-lactam compound but that is capable of producing a β-lactam compound by genetic modification of said microorganism.

2. A microorganism according to claim 1 that is transformed with a polynucleotide involved in the production of said β-lactam compound so as to provide the microorganism with the capacity to produce said β-lactam compound.

3. A microorganism according to claim 2, wherein the polynucleotide involved in the production of said β-lactam compound is a polynucleotide sequence encoding an enzyme of the biosynthetic pathway of said β-lactam compound.

4. A microorganism according to claim 3, wherein the enzyme of the biosynthetic pathway of said β-lactam compound is selected from the group consisting of ACVS, IPNS and AT.

5. A microorganism according to claim 2 that is further transformed with a polynucleotide sequence encoding a protein that has a supportive function in the production of said β-lactam compound.

6. A microorganism according to claim 2 that is a yeast.

7. A yeast according to claim 6, wherein the yeast belongs to a genus selected from the group consisting of Saccharomyces, Candida, Kluyveromyces, Yabadazyma, Pichia, Yarrowia, Neurospora, and Zygosaccharomyces.

8. A process for the production of a β-lactam compound comprising cultivating a microorganism according to claim 2 under conditions conducive to the production of said β-lactam compound, the method optionally comprising a deacylation at position 6 if the β-lactam compound is a penam or at position 7 if the β-lactam compound is a cephem.

9. A process according to claim 8, wherein the β-lactam compound is penicillin G, penicillin V, 6-APA, adipoyl-7-ADCA, adipoyl-7-ACCA, 7-ADCA, 7-ACCA, or 7-ACA.

10. Use of the microorganism of claim 2 to identify factors that influence β-lactam production.
